(19) United States
(12) Patent Application Publication
(10) Pub. No.: US 2019/0233858 A1
(43) Pub. Date: Aug. 1, 2019

(54) METHODS AND MATERIALS FOR THE BIOSYNTHESIS OF COMPOUNDS INVOLVED IN LYSINE METABOLISM AND DERIVATIVES AND COMPOUNDS RELATED THERETO

(71) Applicant: INVISTA NORTH AMERICA S.A.R.L., Wilmington (DE)

(72) Inventors: Massimiliano ZAMPINI, Redcar (GB); Alexander Brett FOSTER, Redcar (GB)

(21) Appl. No.: 16/264,798

(22) Filed: Feb. 1, 2019

Related U.S. Application Data
(60) Provisional application No. 62/624,928, filed on Feb. 1, 2018.

(51) Int. Cl. C12P 13/08; C12N 15/74; C12N 9/88

(52) U.S. Cl. CPC C12P 13/08; C12Y 401/01018; C12N 9/88; C12N 15/74

(57) ABSTRACT

Methods and materials for the biosynthesis of compounds involved in lysine metabolism and/or derivatives and/or compounds related thereto are provided. Also provided are products produced in accordance with these methods and materials.

This patent application claims the benefit of priority from U.S. Provisional Application Ser. No. 62/624,928 filed Feb. 1, 2018, the contents of which are herein incorporated by reference in their entirety.

### FIELD

The present invention relates to biosynthetic methods and materials for the production of compounds involved in lysine metabolism, and/or derivatives thereof and/or other compounds related thereto. The present invention also relates to products biosynthesized or otherwise encompassed by these methods and materials.

Replacement of traditional chemical production processes relying on, for example fossil fuels and/or potentially toxic chemicals, with environmentally friendly (e.g., green chemicals) and/or “cleantech” solutions is being considered, including work to identify building blocks suitable for use in the manufacturing of such chemicals. See, “Conservative evolution and industrial metabolism in Green Chemistry”, Green Chem., 2018, 20, 2171-2191.

Lysine is an economically important amino acid used in various ways, including pharmaceuticals, foods and feed supplements (Mukhtar et al. International Journal of Applied Biology and Forensics 2017 1(2):26-31). L-lysine is one of the nine amino acids which are essential for human and animal nutrition. (Shah et al. Pharm Sci. 2002 15(2):29-35; Anastassiadis. Recent Pat Biotechnol. 2007 1(1):11-24). Its demand has increased significantly in recent years and several hundred thousand tons of this compound are produced annually worldwide mainly by microbial fermentation (Anastassiadis. Recent Pat Biotechnol. 2007 1(1):11-24).

Further, L-lysine is a direct precursor of cadaverine.

Cadaverine is becoming an important C5 platform chemical for the synthesis of polymers including, but not limited to polyamides and nylon formed either via condensation of diacids with diamines or as homopolymers of amino acids, polyurethanes, chelating agents, and additives (Biotechnol Bioeng. 2011 108(1):93-103, Kind and Wittmann Appl Microbiol Biotechnol. 2011 91(5):1287-96, Weichao et al. Green Chemical Engineering 2017 3(3): 308-317). Polymerization of cadaverine in the presence of succinate, adipic acid, or sebacic acid results in production of fully bio-based polyamide products such as, but not limited to PA5,6 and PA5,10 (nylon 5,6 and nylon 5,10) (Kind et al. Metabolic Engineering 2014 25:113-123).

Accordingly, multiple attempts have been made to produce cadaverine from lysine-producing microorganisms (Weichao et al. Green Chemical Engineering 2017 3(3): 308-317).

5-amino valeric acid is another product of lysine with interest as it can polymerize into the polyamide nylon-5 (Liu et al. Sci Rep. 2014 11; 4:5657).

During the last approximately 60 years amino acid production has mainly relied on fermentation process using Corynebacteria (Hermann T. J Biotechnol. 2003 4; 104(1-3):155-72). Corynebacterium glutamicum is one of the main hosts of the amino acid L-lysine, together with Brevibacterium flavum and Brevibacterium lactofermentum (Anastassiadis Recent Pat Biotechnol. 2007 1(1):11-24)). Other important L-lysine producing organisms are engineered Escherichia coli strains (Wang et al. J Ind Microbiol Biotechnol. 43(9):1227-35).

Biosynthetic materials and methods, including organisms having increased production of compounds involved in lysine metabolism, derivatives thereof and compounds related thereto are needed.

### SUMMARY OF THE INVENTION

An aspect of the present invention relates to a process for biosynthesis of compounds involved in lysine metabolism, and/or derivatives thereof and/or compounds related thereto. The process comprises obtaining an organism capable of producing compounds involved in lysine metabolism and derivatives and compounds related thereto, altering the organism, and producing one or more compounds involved in lysine metabolism and derivatives and compounds related thereto in the altered organism as compared to the unaltered organism. In one nonlimiting embodiment, the organism is C. necator or an organism with properties similar thereto. In one nonlimiting embodiment, the organism is altered to express a lysine decarboxylase with or without a PMD exporter system.

In one nonlimiting embodiment, the lysine decarboxylase comprises SEQ ID NO:2 or a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to an amino acid sequence set forth in SEQ ID NO: 2 or a functional fragment thereof. In one nonlimiting embodiment, the lysine decarboxylase is encoded by a nucleic acid sequence comprising SEQ ID NO:1 or a nucleic acid sequence encoding a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO: 1 or a functional fragment thereof. In one nonlimiting embodiment, the lysine decarboxylase comprises cadA EC 4.1.1.18.

In process of the present invention, wherein the organism is altered to express a PMD exporter system, nonlimiting embodiments include PMD antiporter and PMD exporter systems.

In one nonlimiting embodiment, the PMD antiporter comprises E. coli cadB (SEQ ID NO:4) or a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to an amino acid sequence set forth in SEQ ID NO: 4 or a functional fragment thereof. In one nonlimiting embodiment, the PMD antiporter is encoded by a nucleic acid sequence comprising E. coli cadB (SEQ ID NO:3) or a nucleic acid sequence encoding a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO: 3 or a functional fragment thereof.

In one nonlimiting embodiment, the PMD exporter comprises C. glutamicum cg2893 (SEQ ID NO:6) or a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to an amino acid sequence set forth in SEQ ID NO: 6 or a functional fragment thereof.

In one nonlimiting embodiment, the PMD exporter is encoded by a nucleic acid sequence comprising C. glutamicum cg2893 (SEQ ID NO:5) or a nucleic acid sequence encoding a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO: 5 or a functional fragment thereof.

In one nonlimiting embodiment, the nucleic acid sequence is codon optimized for C. necator.

In one nonlimiting embodiment, the organism is further altered to express one or more of a putrescine oxidase, a putrescine transaminase and/or an aldehyde dehydrogenase. In one nonlimiting embodiment, the putrescine oxidase comprises FlavAO EC 1.4.3.10. In one nonlimiting embodiment, the putrescine transaminase comprises ygjG EC 2.6.1.82. In one nonlimiting embodiment the aldehyde dehydrogenase comprises puuC EC 1.2.1.5.

In one nonlimiting embodiment, the organism is further modified to eliminate phaCAB, involved in PHBs production and/or H16-A0006-9 encoding endonucleases thereby improving transformation efficiency.

Another aspect of the present invention relates to an organism altered to produce more compounds involved in lysine metabolism and/or derivatives and compounds related thereto as compared to the unaltered organism. In one nonlimiting embodiment, the organism is C. necator or an organism with properties similar thereto. In one nonlimiting embodiment, the organism is altered to express a lysine decarboxylase with or without a PMD exporter system as disclosed herein.

In one nonlimiting embodiment, the organism is altered with a nucleic acid sequence codon optimized for C. necator.

In one nonlimiting embodiment, the organism is further altered to express one or more of a putrescine oxidase, a putrescine transaminase and/or an aldehyde dehydrogenase as disclosed herein.

In one nonlimiting embodiment, the organism is further modified to eliminate phaCAB, involved in PHBs production and/or H16-A0006-9 encoding endonucleases thereby improving transformation efficiency.

In one nonlimiting embodiment, the organism is altered to express, overexpress, not express or express less of one or more molecules depicted in FIG. 1, 3 or 4. In one nonlimiting embodiment, the molecule(s) comprise a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to an amino acid sequence corresponding to a molecule(s) depicted in FIG. 1A, 1B, 3 or 4, or a functional fragment thereof.

Another aspect of the present invention relates to bio-derived, bio-based, or fermentation-derived products produced from any of the methods and/or altered organisms disclosed herein. Such products include compositions comprising at least one bio-derived, bio-based, or fermentation-derived compound or any combination thereof, as well as bio-derived, bio-based, or fermentation-derived polyamides, nylons, polyurethanes, chelating agents, dietary supplements, proteins, topical medicaments and additives comprising these bio-derived, bio-based, or fermentation-derived compositions or compounds; molded substances obtained by molding the bio-derived, bio-based, or fermentation-derived polymers or resins or the bio-derived, bio-based; bio-derived, bio-based, or fermentation-derived formulations comprising the bio-derived, bio-based, or fermentation-derived compositions or compounds, polyamides, nylons, polyurethanes, chelating agents, dietary supplements, proteins, topical medicaments and additives, or the bio-derived, bio-based, or fermentation-derived molded substances, or any combination thereof; and bio-derived, bio-based, or fermentation-derived semi-solids or non-semi-solid streams comprising the bio-derived, bio-based, or fermentation-derived compositions or compounds, polyamides, nylons, polyurethanes, chelating agents, dietary supplements, proteins, topical medicaments and additives, molded substances or formulations, or any combination thereof.

Another aspect of the present invention relates to a bio-derived, bio-based or fermentation derived product biosynthesized in accordance with the exemplary central metabolism depicted in FIGS. 1A, 1B, 3 and 4.

Another aspect of the present invention relates to exogenous genetic molecules of the altered organisms disclosed herein. In one nonlimiting embodiment, the exogenous genetic molecule comprises a codon optimized nucleic acid sequence encoding a lysine decarboxylase with or without a PMD exporter system. In one nonlimiting embodiment, the exogenous genetic molecule comprises a nucleic acid sequence encoding E. coli cadA (SEQ ID NO:1) or a nucleic acid sequence encoding a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO: 1 or a functional fragment thereof. In one nonlimiting embodiment, the exogenous genetic molecule further comprises a nucleic acid sequence encoding a PMD exporter system. In one nonlimiting embodiment, the exogenous genetic molecule comprises a nucleic acid sequence comprising E. coli cadB (SEQ ID NO:3) or a nucleic acid sequence encoding a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO: 3 or a functional fragment thereof. In one nonlimiting embodiment, the exogenous genetic molecule comprises a nucleic acid sequence comprising C. glutamicum cg2893 (SEQ ID NO:5) or a nucleic acid sequence encoding a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO: 5 or a functional fragment thereof. In one nonlimiting embodiment the exogenous genetic molecule further comprises a nucleic acid sequence encoding one or more of a putrescine oxidase, a putrescine transaminase and/or an aldehyde dehydrogenase. Additional nonlimiting examples of exogenous genetic molecules include expression constructs and synthetic operons of, for example, lysine decarboxylase with or without a PMD exporter system. In some nonlimiting embodiment, the expression constructs or synthetic operons may express a putrescine oxidase, a putrescine transaminase and/or an aldehyde dehydrogenase.

Yet another aspect of the present invention relates to means and processes for use of these means for biosynthesis of compounds involved in lysine metabolism, and/or derivatives thereof and/or compounds related thereto.

### BRIEF DESCRIPTION OF THE FIGURES

FIG. 1A is a schematic of a L-lysine pathway in C. necator H16. The conversion of L-lysine to PMD can occur through the action of a single enzyme, depicted as the lysine decarboxylase encoded by the gene E. coli cadA

FIG. 1B is a schematic of a L-lysine pathway adapted from Weichao et al. (Green Chemical Engineering 2017 3(3): 308-317). Three different variants of the DAP route, referred to as 1^{st}, 2^{nd} and 3^{rd}, are depicted. C. necator H16 uses the 1^{st} variant.

FIG. 2 shows synthesis of cadaverine in E. coli and C. glutamicum. Export of PMD occurs in E. coli through the antiporter cadB. In C. glutamicum export is reported to occur through a permease encoded by the gene cg2893.

FIG. 3 shows enzymatic conversion of cadaverine into the PMD-derivatives 5-amino valeraldehyde and 5-amino 1-pentanol.

FIG. 4 shows 4 exemplary pathways assembled for the detection of the lysine metabolites PMD, 5-amino 1-pentanol and 5-aminovaleric acid.

FIG. 5 shows cadaverine titers at 4-time points in the course of a fed batch cultivation of several C. necator strains. Time points are T1=induction and start of feed, T2=12 hours post feed, T3=36 hours post feed and T4 end of run (˜60 hours). Titers are expressed in ppm (parts per million). Error bars represent the standard deviation.

### DETAILED DESCRIPTION

The present invention provides processes for biosynthesis of compounds involved in lysine metabolism, and/or derivatives thereof, and/or compounds related thereto, as well as organisms altered to increase biosynthesis of compounds involved in lysine metabolism, derivatives thereof and compounds related thereto, exogenous genetic molecules of these altered organisms, and bio-derived, bio-based, or fermentation-derived products biosynthesized or otherwise produced by any of these methods and/or altered organisms.

In one aspect of the present invention, an organism is redirected to produce compounds involved in lysine metabolism, as well as derivatives and compounds related thereto, by alteration of the organism to express a lysine decarboxylase with or without a PMD exporter system. In some embodiments, the organism is further altered to express one or more of a putrescine oxidase, a putrescine transaminase and/or an aldehyde dehydrogenase. Organisms produced in accordance with the present invention are useful in methods for biosynthesizing higher levels of compounds involved in lysine metabolism, derivatives thereof, and compounds related thereto.

For purposes of the present invention, by “compounds involved in lysine metabolism” it is meant to encompass lysine, cadaverine, 5-amino-1-pentanol, 5-aminovaleric acid, and other C5 compounds and aromatic intermediates such as, but not limited to, pipecolate.

For purposes of the present invention, by “derivatives and compounds related thereto” it is meant to encompass compounds derived from the same substrates and/or enzymatic reactions as compounds involved in lysine metabolism, byproducts of these enzymatic reactions and compounds with similar chemical structure including, but not limited to, structural analogs wherein one or more substituents of compounds involved in lysine metabolism are replaced with alternative substituents e.g. other C5 compounds and aromatic derivatives such as pentanedioic acid, 1-butanol, pentanedial, 1,5-pentanediol and 2-oxepanone. This is not intended to be an exhaustive list, merely exemplary.

For purposes of the present invention, by “higher levels of compounds involved in lysine metabolism” it is meant that the altered organisms and methods of the present invention are capable of producing increased levels of compounds involved in lysine metabolism and derivatives and compounds related thereto as compared to the same organism without alteration. In one nonlimiting embodiment, levels are increased by 2-fold or higher.

For compounds containing carboxylic acid groups such as organic monoacids, hydroxyacids, aminoacids and dicarboxylic acids, these compounds may be formed or converted to their ionic salt form when an acidic proton present in the parent compound either is replaced by a metal ion, e.g., an alkali metal ion, an alkaline earth ion, or an aluminum ion; or coordinates with an organic base. Acceptable organic bases include ethanolamine, diethanolamine, triethanolamine, tromethamine, N-methylglucamine, and the like. Acceptable inorganic bases include aluminum hydroxide, calcium hydroxide, potassium hydroxide, sodium carbonate and/or bicarbonate, sodium hydroxide, ammonia and the like. The salt can be isolated as is from the system as the salt or converted to the free acid by reducing the pH to, for example, below the lowest pKa through addition of acid or treatment with an acidic ion exchange resin.

For compounds containing amine groups such as, but not limited to, organic amines, amino acids and diamine, these compounds may be formed or converted to their ionic salt form by addition of an acidic proton to the amine to form the ammonium salt, formed with inorganic acids such as hydrochloric acid, hydrobromic acid, sulfuric acid, nitric acid, phosphoric acid, and the like; or formed with organic acids such as carbonic acid, acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid or muconic acid, and the like. The salt can be isolated as is from the system as a salt or converted to the free amine by raising the pH to, for example, above the highest pKa through addition of base or treatment with a basic ion exchange resin. Acceptable inorganic bases are known in the art and include aluminum hydroxide, calcium hydroxide, potassium hydroxide, sodium carbonate or bicarbonate, sodium hydroxide, and the like.

For compounds containing both amine groups and carboxylic acid groups such as, but not limited to, amino acids, these compounds may be formed or converted to their ionic salt form by either 1) acid addition salts, formed with inorganic acids such as hydrochloric acid, hydrobromic acid, sulfuric acid, nitric acid, phosphoric acid, and the like; or formed with organic acids such as carbonic acid, acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like. Acceptable inorganic bases include aluminum hydroxide, calcium hydroxide, potassium hydroxide, sodium carbonate and/or bicarbonate, sodium hydroxide, and the like, or 2) when an acidic proton present in the parent compound either is replaced by a metal ion, e.g., an alkali metal ion, an alkaline earth ion, or an aluminum ion; or coordinates with an organic base. Acceptable organic bases are known in the art and include ethanolamine, diethanolamine, triethanolamine, trimethylamine, N-methylglucamine, and the like. Acceptable inorganic bases are known in the art and include aluminum hydroxide, calcium hydroxide, potassium hydroxide, sodium carbonate, sodium hydroxide, ammonia and the like. The salt can be isolated as is from the system or converted to the free acid by reducing the pH to, for example, below the pKa through addition of acid or treatment with an acidic ion exchange resin. In one or more aspects of the invention, it is understood that the amino acid salt can be isolated as: i. at low pH, as the ammonium (salt)-free acid form; ii. at high pH, as the amine-carboxylic acid salt form; and/or iii. at neutral or midrange pH, as the free-amine acid form or zwitterion form.

In the process for biosynthesis of compounds involved in lysine metabolism and derivatives and compounds related thereto of the present invention, an organism capable of producing compounds involved lysine metabolism and derivatives and compounds related thereto is obtained. The organism is then altered to produce more compounds involved in lysine metabolism and derivatives and compounds related thereto in the altered organism as compared to the unaltered organism.

In one nonlimiting embodiment, the organism is Cupriavidus necator (C. necator) or an organism with properties similar thereto. A nonlimiting embodiment of the organism is set for at lgcstandards-atcc with the extension .org/products/all/17699.aspx?geo_country=gb#generalinformation of the world wide web.

C. necator (previously called Hydrogenomonas eutrophus, Alcaligenes eutropha, Ralstonia eutropha, and Wautersia eutropha) is a Gram-negative, flagellated soil bacterium of the Betaproteobacteria class. This hydrogen-oxidizing bacterium is capable of growing at the interface of anaerobic and aerobic environments and easily adapts between heterotrophic and autotrophic lifestyles. Sources of energy for the bacterium include both organic compounds and hydrogen. Additional properties of C. necator include microaerophilicity, copper resistance (Makar, N. S. & Casida, L. E. Int. J. of Systematic Bacteriology 1987 37(4): 323-326), bacterial predation (Byrd et al. Can J Microbiol 1985 31:1157-1163; Sillman, C. E. & Casida, L. E. Can J Microbiol 1986 32:760-762; Zeph, L. E. & Casida, L. E. Applied and Environmental Microbiology 1986 52(4):819-823) and polyhydroxybutyrate (PHB) synthesis. In addition, the cells have been reported to be capable of both aerobic and nitrate dependent anaerobic growth. A nonlimiting example of a C. necator organism useful in the present invention is a C. necator of the H16 strain. In one nonlimiting embodiment, a C. necator host of the H16 strain with at least a portion of the phaCAB gene locus knocked out (ΔphaCAB) is used.

In another nonlimiting embodiment, the organism altered in the process of the present invention has one or more of the above-mentioned properties of Cupriavidus necator.

In another nonlimiting embodiment, the organism is selected from members of the genera Ralstonia, Wautersia, Cupriavidus, Alcaligenes, Burkholderia or Pandoraea.

For the process of the present invention, the organism is altered to express a lysine decarboxylase with or without a PMD exporter system.

In one nonlimiting embodiment, the lysine decarboxylase is from E. coli. In one nonlimiting embodiment, the lysine decarboxylase comprises SEQ ID NO:2 or a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to an amino acid sequence set forth in SEQ ID NO: 2 or a functional fragment thereof. In one nonlimiting embodiment, the lysine decarboxylase is encoded by a nucleic acid sequence comprising SEQ ID NO:1 or a nucleic acid sequence encoding a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO: 1 or a functional fragment thereof. In one nonlimiting embodiment, the lysine decarboxylase comprises cadA EC 4.1.1.18.

In process of the present invention, wherein the organism is altered to express a PMD exporter system, nonlimiting embodiments include PMD antiporter and PMD exporter systems.

In one nonlimiting embodiment, the PMD antiporter comprises E. coli cadB (SEQ ID NO:4) or a polypeptide with similar enzymatic activities exhibiting at least about 50%, 60%, 70%, 75%, 80%, 85%, 90%, 91%, 92%, 93%, 94%, 95%, 960, 97%, 98%, 99% or 99.5% sequence identity to an amino acid sequence set forth in SEQ ID NO: 4 or a functional fragment thereof. In one nonlimiting embodiment, the PMD antiporter is encoded by a nucleic acid sequence comprising E. coli cadB (SEQ ID NO:3) or a nucleic acid sequence encoding a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO: 3 or a functional fragment thereof.

In one nonlimiting embodiment, the PMD exporter comprises C. glutamicum cg2893 (SEQ ID NO:6) or a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to an amino acid sequence set forth in SEQ ID NO: 6 or a functional fragment thereof.

In one nonlimiting embodiment, the PMD exporter is encoded by a nucleic acid sequence comprising C. glutamicum cg2893 (SEQ ID NO:5) or a nucleic acid sequence encoding a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO: 5 or a functional fragment thereof.

In some embodiments, the organism is further altered to express one or more of a putrescine oxidase, a putrescine transaminase and/or an aldehyde dehydrogenase.

In one nonlimiting embodiment, the putrescine oxidase is from R. jostii. In one nonlimiting embodiment, the putrescine oxidase comprises SEQ ID NO:8 or a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to an amino acid sequence set forth in SEQ ID NO: 8 or a functional fragment thereof. In one nonlimiting embodiment, the putrescine oxidase is encoded by a nucleic acid sequence comprising SEQ ID NO:7 or a nucleic acid sequence encoding a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO: 7 or a functional fragment thereof. In one nonlimiting embodiment, the putrescine oxidase comprises FlavAO EC 1.4.3.10.

In one nonlimiting embodiment, the putrescine transaminase is from E. coli. In one nonlimiting embodiment, the putrescine transaminase comprises SEQ ID NO:10 or a polypeptide with similar enzymatic activities exhibiting at least about 50%, 60%, 70%, 75%, 80%, 85%, 90%, 91%, 92%, 93%, 94%, 95%, 960, 97%, 98%, 99% or 99.5% sequence identity to an amino acid sequence set forth in SEQ ID NO: 10 or a functional fragment thereof. In one nonlimiting embodiment, the putrescine transaminase is encoded by a nucleic acid sequence comprising SEQ ID NO:9 or a nucleic acid sequence encoding a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO: 9 or a functional fragment thereof. In one nonlimiting embodiment, the putrescine transaminase comprises ygjG EC 2.6.1.82.

In one nonlimiting embodiment, the aldehyde dehydrogenase is from E. coli. In one nonlimiting embodiment, the aldehyde dehydrogenase comprises SEQ ID NO:12 or a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to an amino acid sequence set forth in SEQ ID NO: 12 or a functional fragment thereof. In one nonlimiting embodiment, the aldehyde dehydrogenase is encoded by a nucleic acid sequence comprising SEQ ID NO:11 or a nucleic acid sequence encoding a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO:11 or a functional fragment thereof. In one nonlimiting embodiment, the aldehyde dehydrogenase comprises puuC EC 1.2.1.5.

In one nonlimiting embodiment, the nucleic acid sequence is codon optimized for C. necator.

In one nonlimiting embodiment, the organism is further modified to eliminate phaCAB, involved in PHBs production and/or H16-A0006-9 encoding endonucleases thereby improving transformation efficiency as described in U.S. patent application Ser. No. 15/717,216, teachings of which are incorporated herein by reference.

In the process of the present invention, the altered organism is then subjected to conditions wherein compounds involved in lysine metabolism and derivatives and compounds related thereto are produced.

In the process described herein, a fermentation strategy can be used that entails anaerobic, micro-aerobic or aerobic cultivation. A fermentation strategy can entail nutrient limitation such as nitrogen, phosphate or oxygen limitation.

Under conditions of nutrient limitation a phenomenon known as overflow metabolism (also known as energy spilling, uncoupling or spillage) occurs in many bacteria (Russell, 2007). In growth conditions in which there is a relative excess of carbon source and other nutrients (e.g. phosphorous, nitrogen and/or oxygen) are limiting cell growth, overflow metabolism results in the use of this excess energy (or carbon), not for biomass formation but for the excretion of metabolites, typically organic acids. In Cupriavidus necator a modified form of overflow metabolism occurs in which excess carbon is sunk intracellularly into the storage carbohydrate polyhydroxybutyrate (PHB). In strains of C. necator which are deficient in PHB synthesis this overflow metabolism can result in the production of extracellular overflow metabolites. The range of metabolites that have been detected in PHB deficient C. necator strains include acetate, acetone, butanoate, cis-aconitate, citrate, ethanol, fumarate, 3-hydroxybutanoate, propan-2-ol, malate, methanol, 2-methyl-propanoate, 2-methyl-butanoate, 3-methyl-butanoate, 2-oxoglutarate, meso-2,3-butanediol, acetoin, DL-2,3-butanediol, 2-methylpropan-1-ol, propan-1-ol, lactate 2-oxo-3-methylbutanoate, 2-oxo-3-methylpentanoate, propanoate, succinate, formic acid and pyruvate. The range of overflow metabolites produced in a particular fermentation can depend upon the limitation applied (e.g. nitrogen, phosphate, oxygen), the extent of the limitation, and the carbon source provided (Schlegel, H. G. & Vollbrecht, D. Journal of General Microbiology 1980 117:475-481; Steinbüchel, A. & Schlegel, H. G. Appl Microbiol Biotechnol 1989 31: 168; Vollbrecht et al. Eur J Appl Microbiol Biotechnol 1978 6:145-155; Vollbrecht et al. European J. Appl. Microbiol. Biotechnol. 1979 7: 267; Vollbrecht, D. & Schlegel, H. G. European J. Appl. Microbiol. Biotechnol. 1978 6: 157; Vollbrecht, D. & Schlegel, H. G. European J. Appl. Microbiol. Biotechnol. 1979 7: 259).

Applying a suitable nutrient limitation in defined fermentation conditions can thus result in an increase in the flux through a particular metabolic node. The application of this knowledge to C. necator strains genetically modified to produce desired chemical products via the same metabolic node can result in increased production of the desired product.

A cell retention strategy using a ceramic hollow fiber membrane can be employed to achieve and maintain a high cell density during fermentation. The principal carbon source fed to the fermentation can derive from a biological or non-biological feedstock. The biological feedstock can be, or can derive from, monosaccharides, disaccharides, lignocellulose, hemicellulose, cellulose, lignin, levulinic acid and formic acid, triglycerides, glycerol, fatty acids, agricultural waste, condensed distillers' solubles or municipal waste. The non-biological feedstock can be, or can derive from, natural gas, syngas, CO₂/H₂, CO, H₂, O₂, methanol, ethanol, non-volatile residue (NVR) a caustic wash waste stream from cyclohexane oxidation processes or waste stream from a chemical industry such as, but not limited to a carbon black industry or a hydrogen-refining industry, or petrochemical industry.

In one nonlimiting embodiment, at least one of the enzymatic conversions of the production method comprises gas fermentation within the altered Cupriavidus necator host, or a member of the genera Ralstonia, Wautersia, Alcaligenes, Burkholderia and Pandoraea, and other organism having one or more of the above-mentioned properties of Cupriavidus necator. In this embodiment, the gas fermentation may comprise at least one of natural gas, syngas, CO₂/H₂, CO, H₂, O₂, methanol, ethanol, non-volatile residue, caustic wash from cyclohexane oxidation processes, or waste stream from a chemical industry such as, but not limited to a carbon black industry or a hydrogen-refining industry, or petrochemical industry. In one nonlimiting embodiment, the gas fermentation comprises CO₂/H₂.

The methods of the present invention may further comprise recovering produced compounds involved in lysine metabolism or derivatives or compounds related thereto. Once produced, any method can be used to isolate the compound or compounds involved in lysine metabolism or derivatives or compounds related thereto.

The present invention also provides altered organisms capable of biosynthesizing increased amounts of compounds involved in lysine metabolism and derivatives and compounds related thereto as compared to the unaltered organism. In one nonlimiting embodiment, the altered organism of the present invention is a genetically engineered strain of Cupriavidus necator capable of producing compounds involved in lysine metabolism and derivatives and compounds related thereto. In another nonlimiting embodiment, the organism to be altered is selected from members of the genera Ralstonia, Wautersia, Alcaligenes, Cupriavidus, Burkholderia and Pandoraea, and other organisms having one or more of the above-mentioned properties of Cupriavidus necator. In one nonlimiting embodiment, the present invention relates to a substantially pure culture of the altered organism capable of producing compounds involved in lysine metabolism and derivatives and compounds related thereto via lysine decarboxylase.

As used herein, a “substantially pure culture” of an altered organism is a culture of that microorganism in which less than about 40% (i.e., less than about 35%; 30%; 25%; 20%; 15%; 10%; 5%; 2%; 1%; 0.5%; 0.25%; 0.1%; 0.01%; 0.001%; 0.0001%; or even less) of the total number of viable cells in the culture are viable cells other than the altered microorganism, e.g., bacterial, fungal (including yeast), mycoplasmal, or protozoan cells. The term “about” in this context means that the relevant percentage can be 15% of the specified percentage above or below the specified percentage. Thus, for example, about 20% can be 17% to 23%. Such a culture of altered microorganisms includes the cells and a growth, storage, or transport medium. Media can be liquid, semi-solid (e.g., gelatinous media), or frozen. The culture includes the cells growing in the liquid or in/on the semi-solid medium or being stored or transported in a storage or transport medium, including a frozen storage or transport medium. The cultures are in a culture vessel or storage vessel or substrate (e.g., a culture dish, flask, or tube or a storage vial or tube).

Altered organisms of the present invention comprise at least one genome-integrated synthetic operon encoding an enzyme.

In one nonlimiting embodiment, the altered organism is produced by integration of a synthetic operon encoding with or without a PMD exporter.

In one nonlimiting embodiment, the lysine decarboxylase is from E. coli. In one nonlimiting embodiment, the lysine decarboxylase comprises SEQ ID NO:2 or a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to an amino acid sequence set forth in SEQ ID NO: 2 or a functional fragment thereof. In one nonlimiting embodiment, the lysine decarboxylase is encoded by a nucleic acid sequence comprising SEQ ID NO:1 or a nucleic acid sequence encoding a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO: 1 or a functional fragment thereof. In one nonlimiting embodiment, the lysine decarboxylase comprises cadA EC 4.1.1.18.

In organisms of the present invention altered to express a PMD exporter system, nonlimiting embodiments include PMD antiporter and PMD exporter systems.

In one nonlimiting embodiment, the PMD antiporter comprises E. coli cadB (SEQ ID NO:4) or a polypeptide with similar enzymatic activities exhibiting at least about 50%, 60%, 70%, 75%, 80%, 85%, 90%, 91%, 92%, 93%, 94%, 95%, 960, 97%, 98%, 99% or 99.5% sequence identity to an amino acid sequence set forth in SEQ ID NO: 4 or a functional fragment thereof. In one nonlimiting embodiment, the PMD antiporter is encoded by a nucleic acid sequence comprising E. coli cadB (SEQ ID NO:3) or a nucleic acid sequence encoding a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO: 3 or a functional fragment thereof.

In one nonlimiting embodiment, the PMD exporter comprises C. glutamicum cg2893 (SEQ ID NO:6) or a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to an amino acid sequence set forth in SEQ ID NO: 6 or a functional fragment thereof. In one nonlimiting embodiment, the PMD exporter is encoded by a nucleic acid sequence comprising C. glutamicum cg2893 (SEQ ID NO:5) or a nucleic acid sequence encoding a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO: 5 or a functional fragment thereof.

In some embodiments, the organism is further altered to express one or more of a putrescine oxidase, a putrescine transaminase and/or an aldehyde dehydrogenase.

In one nonlimiting embodiment, the putrescine oxidase is from R. jostii. In one nonlimiting embodiment, the putrescine oxidase comprises SEQ ID NO:8 or a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to an amino acid sequence set forth in SEQ ID NO: 8 or a functional fragment thereof. In one nonlimiting embodiment, the putrescine oxidase is encoded by a nucleic acid sequence comprising SEQ ID NO:7 or a nucleic acid sequence encoding a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO: 7 or a functional fragment thereof. In one nonlimiting embodiment, the putrescine oxidase comprises FlavAO EC 1.4.3.10.

In one nonlimiting embodiment, the putrescine transaminase is from E. coli. In one nonlimiting embodiment, the putrescine transaminase comprises SEQ ID NO:10 or a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to an amino acid sequence set forth in SEQ ID NO: 10 or a functional fragment thereof. In one nonlimiting embodiment, the putrescine transaminase is encoded by a nucleic acid sequence comprising SEQ ID NO:9 or a nucleic acid sequence encoding a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO: 9 or a functional fragment thereof. In one nonlimiting embodiment, the putrescine transaminase comprises ygjG EC 2.6.1.82.

In one nonlimiting embodiment, the aldehyde dehydrogenase is from E. coli. In one nonlimiting embodiment, the aldehyde dehydrogenase comprises SEQ ID NO:12 or a polypeptide with similar enzymatic activities exhibiting at least about 50%, 60%, 70%, 75%, 80%, 85%, 90%, 91%, 92%, 93%, 94%, 95%, (CO, 97%, 98%, 99% or 99.5% sequence identity to an amino acid sequence set forth in SEQ ID NO: 12 or a functional fragment thereof. In one nonlimiting embodiment, the aldehyde dehydrogenase is encoded by a nucleic acid sequence comprising SEQ ID NO:11 or a nucleic acid sequence encoding a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO:11 or a functional fragment thereof. In one nonlimiting embodiment, the aldehyde dehydrogenase comprises puuC EC 1.2.1.5.

In one nonlimiting embodiment, the nucleic acid sequence is codon optimized for C. necator.

In one nonlimiting embodiment, the organism is further modified to eliminate phaCAB, involved in PHBs production and/or H16-A0006-9 encoding endonucleases thereby improving transformation efficiency.

The percent identity (and/or homology) between two amino acid sequences as disclosed herein can be determined as follows. First, the amino acid sequences are aligned using the BLAST 2 Sequences (B12seq) program from the stand-alone version of BLAST containing BLASTP version 2.0.14. This stand-alone version of BLAST can be obtained from the U.S. government's National Center for Biotechnology Information web site (www with the extension ncbi.nlm.nih.gov). Instructions explaining how to use the B12seq program can be found in the readme file accompanying BLASTZ. B12seq performs a comparison between two amino acid sequences using the BLASTP algorithm. To compare two amino acid sequences, the options of B12seq are set as follows: -i is set to a file containing the first amino acid sequence to be compared (e.g., C:\seql.txt); -j is set to a file containing the second amino acid sequence to be compared (e.g., C:\seq2.txt); -p is set to blastp; -o is set to any desired file name (e.g., C:\output.txt); and all other options are left at their default setting. For example, the following command can be used to generate an output file containing a comparison between two amino acid sequences: C:\B12seq-i c:\seql.txt-j c:\seq2.txt-p blastp-o c:\output.txt. If the two compared sequences share homology (identity), then the designated output file will present those regions of homology as aligned sequences. If the two compared sequences do not share homology (identity), then the designated output file will not present aligned sequences. Similar procedures can be followed for nucleic acid sequences except that blastn is used.

Once aligned, the number of matches is determined by counting the number of positions where an identical amino acid residue is presented in both sequences. The percent identity (homology) is determined by dividing the number of matches by the length of the full-length polypeptide amino acid sequence followed by multiplying the resulting value by 100. It is noted that the percent identity (homology) value is rounded to the nearest tenth. For example, 90.11, 90.12, 90.13, and 90.14 is rounded down to 90.1, while 90.15, 90.16, 90.17, 90.18, and 90.19 is rounded up to 90.2. It also is noted that the length value will always be an integer.

It will be appreciated that a number of nucleic acids can encode a polypeptide having a particular amino acid sequence. The degeneracy of the genetic code is well known to the art; i.e., for many amino acids, there is more than one nucleotide triplet that serves as the codon for the amino acid. For example, codons in the coding sequence for a given enzyme can be modified such that optimal expression in a particular species (e.g., bacteria or fungus) is obtained, using appropriate codon bias tables for that species.

Functional fragments of any of the polypeptides or nucleic acid sequences described herein can also be used in the methods and organisms disclosed herein. The term “functional fragment” as used herein refers to a peptide or fragment of a polypeptide or a nucleic acid sequence fragment encoding a peptide fragment of a polypeptide that has at least about 25% (e.g., at least about 30%; 40%; 50%; 60%; 70%; 75%; 80%; 85%; 90%; 95%; 98%; 99%; 100%; or even greater than 100%) of the activity of the corresponding mature, full-length, polypeptide. The functional fragment can generally, but not always, be comprised of a continuous region of the polypeptide, wherein the region has functional activity.

Functional fragments may range in length from about 10% up to 99% (inclusive of all percentages in between) of the original full-length sequence.

This document also provides (i) functional variants of the enzymes used in the methods of the document and (ii) functional variants of the functional fragments described above. Functional variants of the enzymes and functional fragments can contain additions, deletions, or substitutions relative to the corresponding wild-type sequences. Enzymes with substitutions will generally have not more than 50 (e.g., not more than one, two, three, four, five, six, seven, eight, nine, ten, 12, 15, 20, 25, 30, 35, 40, or 50) amino acid substitutions (e.g., conservative substitutions). This applies to any of the enzymes described herein and functional fragments. A conservative substitution is a substitution of one amino acid for another with similar characteristics. Conservative substitutions include substitutions within the following groups: valine, alanine and glycine; leucine, valine, and isoleucine; aspartic acid and glutamic acid; asparagine and glutamine; serine, cysteine, and threonine; lysine and arginine; and phenylalanine and tyrosine. The nonpolar hydrophobic amino acids include alanine, leucine, isoleucine, valine, proline, phenylalanine, tryptophan and methionine. The polar neutral amino acids include glycine, serine, threonine, cysteine, tyrosine, asparagine and glutamine. The positively charged (basic) amino acids include arginine, lysine and histidine. The negatively charged (acidic) amino acids include aspartic acid and glutamic acid. Any substitution of one member of the above-mentioned polar, basic or acidic groups by another member of the same group can be deemed a conservative substitution. By contrast, nonconservative substitution is a substitution of one amino acid for another with dissimilar characteristics.

Deletion variants can lack one, two, three, four, five, six, seven, eight, nine, ten, 11, 12, 13, 14, 15, 16, 17, 18, 19, or 20 amino acid segments (of two or more amino acids) or non-contiguous single amino acids. Additions (addition variants) include fusion proteins containing: (a) any of the enzymes described herein or a fragment thereof; and (b) internal or terminal (C or N) irrelevant or heterologous amino acid sequences. In the context of such fusion proteins, the term “heterologous amino acid sequences” refers to an amino acid sequence other than (a). A heterologous sequence can be, for example a sequence used for purification of the recombinant protein (e.g., FLAG, polyhistidine (e.g., hexahistidine), hemagluttanin (HA), glutathione-S-transferase (GST), or maltose binding protein (MBP)). Heterologous sequences also can be proteins useful as detectable markers, for example, luciferase, green fluorescent protein (GFP), or chloramphenicol acetyl transferase (CAT). In some embodiments, the fusion protein contains a signal sequence from another protein. In certain host cells (e.g., yeast host cells), expression and/or secretion of the target protein can be increased through use of a heterologous signal sequence. In some embodiments, the fusion protein can contain a carrier (e.g., KLH) useful, e.g., in eliciting an immune response for antibody generation) or ER or Golgi apparatus retention signals. Heterologous sequences can be of varying length and in some cases can be a longer sequences than the full-length target proteins to which the heterologous sequences are attached.

Endogenous genes of the organisms altered for use in the present invention also can be disrupted to prevent the formation of undesirable metabolites or prevent the loss of intermediates in the pathway through other enzymes acting on such intermediates. In one nonlimiting embodiment, the organism is further modified to eliminate phaCAB, involved in PHBs production and/or H16-A0006-9 encoding endonucleases thereby improving transformation efficiency.

Thus, as described herein, altered organisms can include exogenous nucleic acids encoding a lysine decarboxylase, a PMD exporter system, a putrescine oxidase, a putrescine transaminase and/or an aldehyde dehydrogenase, as described herein, as well as modifications to endogenous genes.

The term “exogenous” as used herein with reference to a nucleic acid (or a protein) and an organism refers to a nucleic acid that does not occur in (and cannot be obtained from) a cell of that particular type as it is found in nature or a protein encoded by such a nucleic acid. Thus, a non-naturally-occurring nucleic acid is considered to be exogenous to a host or organism once in or utilized by the host or organism. It is important to note that non-naturally-occurring nucleic acids can contain nucleic acid subsequences or fragments of nucleic acid sequences that are found in nature provided the nucleic acid as a whole does not exist in nature. For example, a nucleic acid molecule containing a genomic DNA sequence within an expression vector is non-naturally-occurring nucleic acid, and thus is exogenous to a host cell once introduced into the host, since that nucleic acid molecule as a whole (genomic DNA plus vector DNA) does not exist in nature. Thus, any vector, autonomously replicating plasmid, or virus (e.g., retrovirus, adenovirus, or herpes virus) that as a whole does not exist in nature is considered to be non-naturally-occurring nucleic acid. It follows that genomic DNA fragments produced by PCR or restriction endonuclease treatment as well as cDNAs are considered to be non-naturally-occurring nucleic acid since they exist as separate molecules not found in nature. It also follows that any nucleic acid containing a promoter sequence and polypeptide-encoding sequence (e.g., cDNA or genomic DNA) in an arrangement not found in nature is non-naturally-occurring nucleic acid. A nucleic acid that is naturally-occurring can be exogenous to a particular host microorganism. For example, an entire chromosome isolated from a cell of yeast x is an exogenous nucleic acid with respect to a cell of yeast y once that chromosome is introduced into a cell of yeast y.

In contrast, the term “endogenous” as used herein with reference to a nucleic acid (e.g., a gene) (or a protein) and a host refers to a nucleic acid (or protein) that does occur in (and can be obtained from) that particular host as it is found in nature. Moreover, a cell “endogenously expressing” a nucleic acid (or protein) expresses that nucleic acid (or protein) as does a host of the same particular type as it is found in nature. Moreover, a host “endogenously producing” or that “endogenously produces” a nucleic acid, protein, or other compound produces that nucleic acid, protein, or compound as does a host of the same particular type as it is found in nature.

The present invention also provides exogenous genetic molecules of the nonnaturally occurring organisms disclosed herein such as, but not limited to, codon optimized nucleic acid sequences, expression constructs and/or synthetic operons.

In one nonlimiting embodiment, the exogenous genetic molecule comprises a codon optimized nucleic acid sequence encoding a lysine decarboxylase, a PMD exporter system, a putrescine oxidase, a putrescine transaminase and/or an aldehyde dehydrogenase as disclosed herein. In one nonlimiting embodiment, the nucleic acid sequence is codon optimized for C. necator. Additional nonlimiting examples of exogenous genetic molecules include expression constructs of, for example, a lysine decarboxylase, a PMD exporter system, a putrescine oxidase, a putrescine transaminase and/or an aldehyde dehydrogenase as disclosed herein.

Also provided by the present invention are compounds involved in lysine metabolism and derivatives and compounds related thereto bioderived from an altered organism according to any of methods described herein.

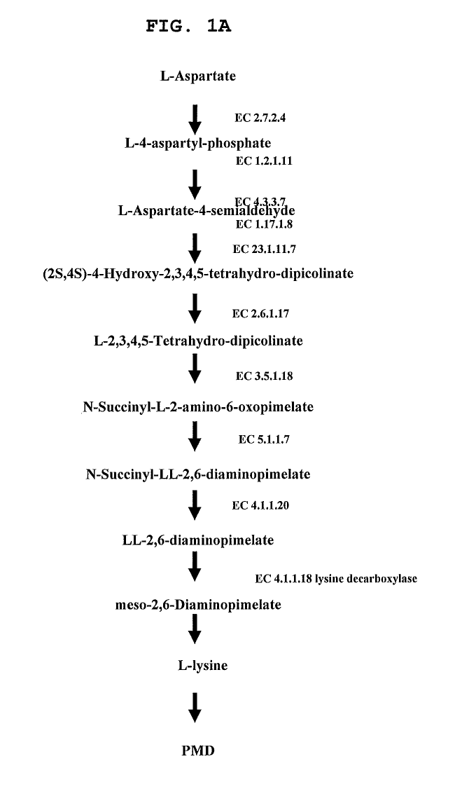

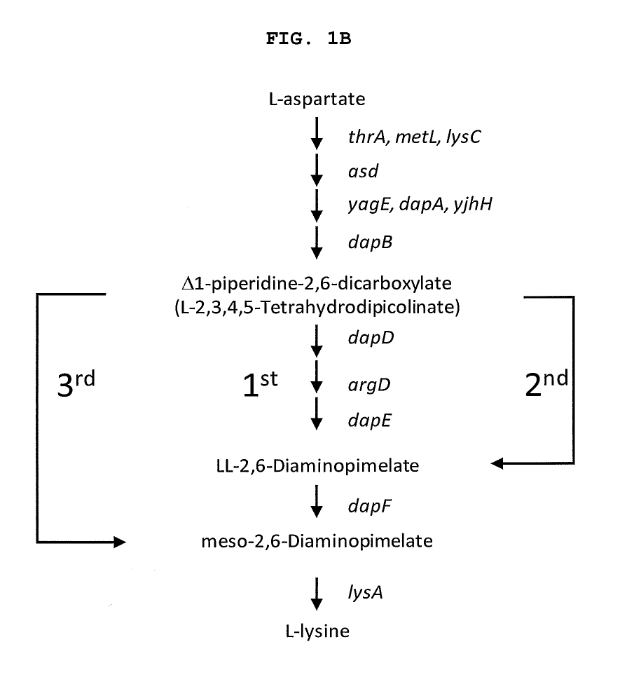

Further, the present invention relates to means and processes for use of these means for biosynthesis of compounds involved in lysine metabolism and derivatives and compounds related thereto. Nonlimiting examples of such means include altered organisms and exogenous genetic molecules as described herein as well as any of the molecules as depicted in FIGS. 1A, 1B, 3 and 4.

In addition, the present invention provides bio-derived, bio-based, or fermentation-derived products produced using the methods and/or altered organisms disclosed herein. In one nonlimiting embodiment, a bio-derived, bio-based or fermentation derived product is produced in accordance with the exemplary central metabolism depicted in FIG. 1A, 1B, 3, or 4. Examples of such products include, but are not limited to, compositions comprising at least one bio-derived, bio-based, or fermentation-derived compound or any combination thereof, as well as polyamides, nylons, polyurethanes, chelating agents, dietary supplements, proteins, topical medicaments and additives, molded substances, formulations and semi-solid or non-semi-solid streams comprising one or more of the bio-derived, bio-based, or fermentation-derived compounds or compositions, combinations or products thereof.

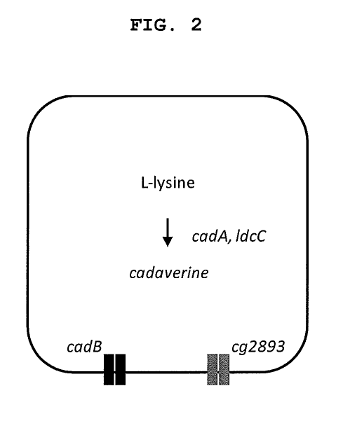

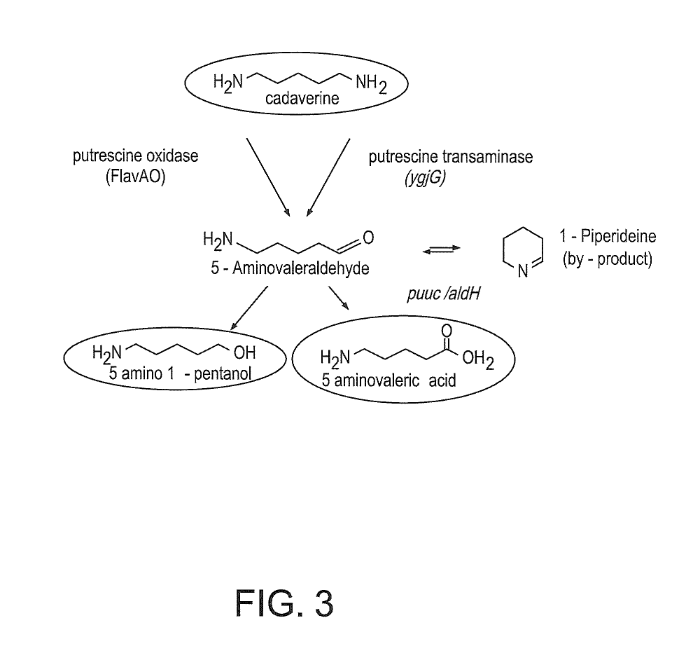

The carbon flux through the L-lysine (FIG. 1A and FIG. 1B) pathway in C. necator H16 (ΔphaCAB ΔA0006-9) was assessed. For these studies, the lysine metabolites PMD (cadaverine) (FIG. 2), 5-amino-1-pentanol and 5-aminovaleric acid (FIG. 3) were monitored.

Two pathways have been described in literature for the biosynthesis of L-lysine: the diaminopimelicacid (DAP) route (bacteria/plants), and the α-aminoadipic acid pathway (fungi/archaea) (Weichao et al. Green Chemical Engineering 2017 3(3): 308-317). The DAP pathway exists in three further variants, all of them leading to the synthesis of the same metabolite: meso-2,6-diaminopimelate (FIG. 1B). The most common variant (FIG. 1B, 1^{st}-reported genes are those found in E. coli) is found in eubacteria (including C. necator H16), fungi, plants and archaea. The second variant (FIG. 1B, 2^{nd}) is typical of Bacillus species, and the third (FIG. 1B, 3^{rd}) is found mainly in the strong L-lysine producer C. glutamicum, and few other species.

The export of L-lysine in the culture medium has been investigated in L-lysine producers such as E. coli and C. glutamicum. The main transporters in these two microorganisms are the products of the genes cadB and cg2893, respectively.

In several bacteria species, the conversion of L-lysine into PMD (FIG. 2) is achieved by the action of a lysine decarboxylase. In E. coli this enzyme is encoded by the genes cadA (Kind and Wittmann. Appl Microbiol Biotechnol. 2011 91(5):1287-96, Kind et al. Metab Eng. 2014 25:113-23, Kwak et al. Biotechnol Biofuels 2017 21; 10:20) and ldcC. LdcC has optimum activity at pH 7.6 while CadA has been reported to be most active at pH 5.6. Additionally, CadA has greater thermal stability and higher enzymatic activity (Weichao et al. Green Chemical Engineering 2017 3(3): 308-317).

PMD is a natural product synthesized in E. coli during anaerobic growth in the presence of L-lysine and at a low pH, as an adaptive response to environmental acidic conditions or in the absence of putrescine biosynthesis. Its export from the cell is important to reduce toxicity and simplify purification.

In E. coli, cadaverine export occurs through the antiporter CadB (Tomitori et al. Amino Acids 2012 42(2-3):733-40), a membrane protein importing L-lysine in exchange for PMD (FIG. 2). In Corynebacterium glutamicum a putative strong PMD exporter (permease) has been described and successfully employed for the biosynthesis of cadaverine from this microorganism (Kind and Wittmann. Appl Microbiol Biotechnol. 2011 91(5):1287-96, Kind et al. Metab Eng. 2014 25:113-23). This exporter is a protein encoded by the gene cg2893 (FIG. 2).

To avoid potential toxicity effects associated with overexpression of cadaverine, production of two derivatives 5-amino-1-pentanol and 5-amino valeric acid were also examined (FIG. 3). In this assessment, cadaverine is converted to 5-amino valeraldehyde through two independent enzymatic routes, (i) the action of a broad-specificity amine oxidase (here indicated as FlavAO) or (ii) the transamination by a broad-specificity amine transaminase (ygjG). The further conversion of the reactive 5-amino valeraldehyde into the corresponding alcohol and carboxylic acid may be stimulated by the expression of an additional aldehyde dehydrogenase (such as that encoded by puuC).

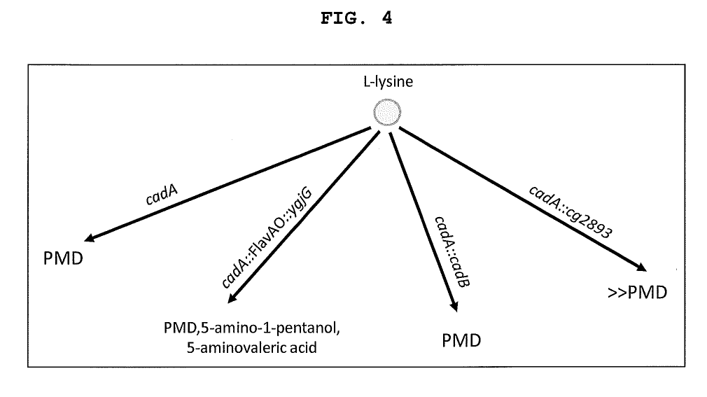

Accordingly, the following four pathways were assembled (FIG. 4) into a kanamycin-resistance pBBR1-derivative, and the various operons were expressed under the control of the L-arabinose-inducible promoter PBAD.

1) cadA

2) cadA::FlavAO::ygjG

3) cadA::cadB

4) cadA::cg2893

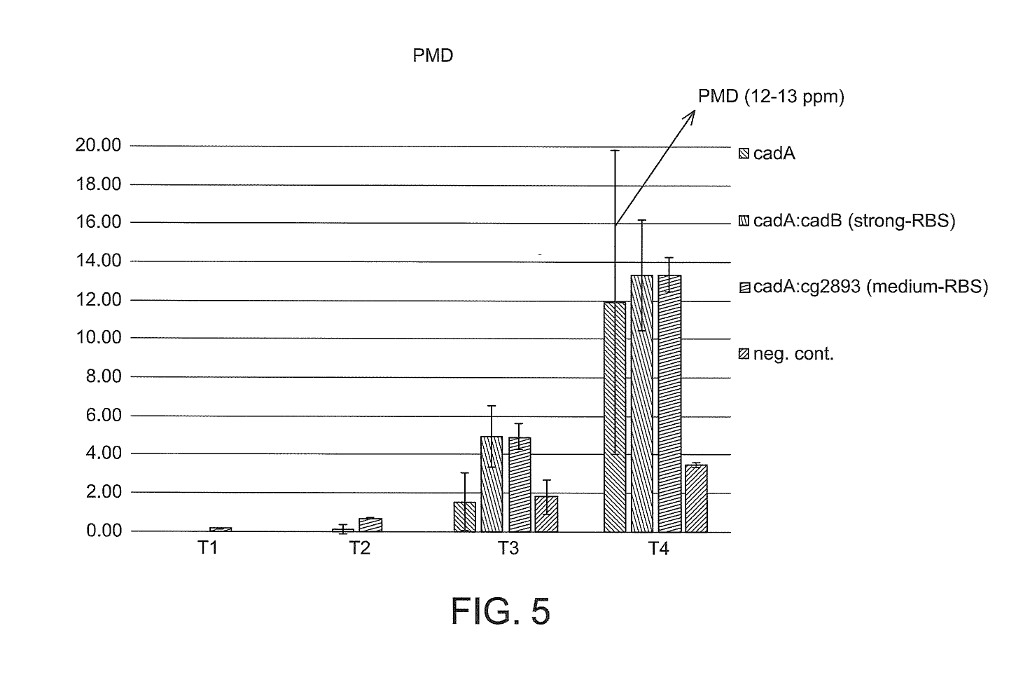

Results from experiments performed through the fermentation platform Ambr15f system are shown in FIG. 5.

The Ambr15f is a small scale (15 ml), moderately high throughput (24 vessels) semi-automated fermentation platform. It encompasses many of the characteristics of a continuous stirrer tank reactor or CSTR such as temperature, pH and DO control, media feeding (exponential, linear, constant) as well as the ability to feed air, oxygen and nitrogen gases.

Strains were screened in the Ambr15f under fed batch conditions with fructose as the carbon source. Several samples were taken over the course of the batch and feeding portions of growth, and target molecules accessed via LCMS.

This screening methodology allowed productivity to be quantified in high cell density cultures under stringent control, demonstrating early, the potential for pathways to achieve high titers in a simple, scalable process.

Four strains expressing respectively cadA, cadA::cadB(strong-Ribosome binding site (RBS)), cadA:cg2893(medium-RBS) and the negative control were tested under fed batch conditions with stringent controls as described in the material and methods. Peak titers achieved were as high as approximately 15 ppm (FIG. 5). Lysine was also produced in many of the cultures to a similar level. These strains were induced and feeds started, approximately 12 hours after inoculation. Most DO (dissolved oxygen) traces are consistent with the growth profile expected i.e. cultures reached and maintained a DO of 10%. Similarly, pH control was consistent throughout, and had no aberrant effects on production. On removing the vessels it was apparent that a strain expressing cadA only was very low in regard volume.

PMD also accumulated over time (increasing from T1 to T4) up to about 12-13 ppm in T4, also after the end of the feed phase (T3).

The following section provides further illustration of the methods and materials of the present invention. These Examples are illustrative only and are not intended to limit the scope of the invention in any way.

### Examples

### Electroporation

Electrocompetent C. necator cells were prepared following a standard procedure.

### Bacterial Strains and Plasmids

All assays were performed in Cupriavidus necator H16 ΔphaCAB. This strain was transformed with constructs derived from plasmid pBBR1, as shown in Table 1, with genes as described in Table 2. All vectors were assembled by standard cloning techniques such as described, for example in Green and Sambrook, Molecular Cloning, A Laboratory Manual, Nov. 18, 2014.

TABLE 1
Species/genes (on plasmid)

C. necator H16, no genes, negative control
C. necator H16, cadA
C. necator H16, cadA, FlavAO, ygjG
C. necator H16, cadA, cadB (strong RBS)
C. necator H16, cadA, cadB (medium-strength RBS)
C. necator H16, cadA, cadB (weak RBS)
C. necator H16, cadA, cg2893 (strong RBS) (never tested)**
C. necator H16, cadA, cg2893 (medium-strength RBS)
C. necator H16, cadA, cg2893 (weak RBS)
pJ-Amp-low:: cadA
pJ-Amp-low::cadB(strong RBS)
pJ-Amp-low::cadB(medium-strength RBS)
pJ-Amp-low::cadB(weak RBS)
pJ-Amp-high::FlavAO
pJ-Amp-high::ygjG
pJ-Amp-high::puuC
pJ-Amp-low::cg2893
*These plasmids are hosted in E. coli NEB5a strains. For the various assays they were transformed into the C. necator H16 strains (all ΔphaCAB, Δ0006-9 derivatives) described in this table

TABLE 2

| Gene | | | | NCBI/Uniprot |
|---|---|---|---|---|
| name | function | species | Reference | ID |
| cadA | lysine | E. coli | Kwak et al. 2017 | Uniprot: |
| | decarboxylase | | Schneider & | P0A9H3 |
| | | | Wendisch. 2010 | |
| cadB | PMD | E. coli | Tomitori | Uniprot: |
| | antiporter | | et al. 2012 | P0AAE8 |
| cg2896 | PMD | Corynebacterium | Kind et al. 2011 | GenBank: |
| | exporter | glutamicum | Kind et al. 2014 | WP_011015248.1 |
| RHA1_ro05606 | putrescine | Rhodococcus | Foster et al. 2013 | GenBank: |
| (“FlavAO”) | oxidase | jostii | Foster et al. 2013b | ABG97386.1 |
| ygjG | putrescine | E. coli | Samsonova et al | GenBank: |
| | transaminase | | 2003 | NP_417544.5 |
| puuC | Aldehyde | E. coli | Jo et al. 2008. | GenBank: |
| | dehydrogenase | | | AAC74382.1 |

### Preparation of Samples for Mass-Spectrometry Liquid-Chromatography

Pre-cultures (10 ml) were prepared in TSB medium using standard procedures. Cells were subsequently washed in a defined minimal media before inoculation. After growth upon the defined minimal media with fructose as the carbon source, cells were induced with L-Arabinose. Samples were then purified from cells and proteins by subsequent (i) centrifugation, (ii) filtration (0.22 μm) and (iii) ultrafiltration (96-well filters, cut-off 10 kDa). The clarified supernatants were analyzed through mass-spectrometry.

### Mass-Spectrometry Analysis

Cadaverine, lysine, 5-aminovaleric acid and 5-aminopentanol concentrations of samples were determined by liquid chromatography-mass spectrometry (LC-MS). For analysis of extracellular compounds, the broth samples were centrifuged and the resulting supernatants were diluted in 90% LC-MS grade acetonitrile/10% LC-MS grade water between 10- and 100-fold, depending upon anticipated analyte concentration.

LC-MS was performed using an Agilent Technologies (Santa Clara, Calif., USA) 1290 Series Infinity HPLC system, coupled to an Agilent 6530 Series Q-TOF mass spectrometer. Manufacturer instructions were followed using a BEH Amide UPLC column: 2.1 mm diameter×50 mm length×1.7 μm particle size (Waters, Milford, Mass., USA)

External standard curves were used for quantitation. Calibration levels of 0.01, 0.02, 0.05, 0.1, 0.2, 0.5, 1, 2, 5 and 10 μg/ml were constructed in a matrix-matched solution, typically the blank medium, diluted to the same level as the samples in acetonitrile.

### Fermentation Assays in the Ambr15f System

Seed Train

Cultures were first incubated overnight and then sub-cultured and further incubated for 16 hours. These cultures were used as a direct inoculum for the fermentation fed batch cultures.

Fermentation

The Sartorius Ambr15F platform was used to screen pathway strains in a fed batch mode of operation. This system allowed control of multiple variables such as dissolved oxygen and pH. Typically, the following process conditions were standardized and run following manufacturer instructions.

Sample Preparation for Analysis

Samples volumes were usually 500 μl and from this 100 μl was used for OD₆₀₀ determination and 400 μl processed for analysis. Processing involved centrifugation to pellet cells (2000 g, 30 minutes) and passage of supernatant through a 0.2 μm filter. The clarified supernatant was then diluted first, or injected directly into the analytical machine.

### Sequence Information for Sequences in Sequence Listing

TABLE 3

| SEQ ID NO: | Sequence Description |
|---|---|
| 1 | cadA DNA sequence C. necator-optimized |
| 2 | cadA Translated protein |
| 3 | cadB DNA sequence C. necator-optimized |
| 4 | cadB Translated protein |
| 5 | Cg2893 DNA sequence C. necator-optimized |
| 6 | Cg2893 Translated protein |
| 7 | RHA1_ro05606 (“FlavAO”) DNA sequence |
| | C. necator-optimized |
| 8 | RHA1_ro05606 (“FlavAO”) Translated protein |
| 9 | ygjG DNA sequence C. necator-optimized |
| 10 | ygjG Translated protein |
| 11 | puuC DNA sequence C. necator-optimized |
| 12 | puuC Translated protein |

1. A process for biosynthesis of compounds involved in lysine metabolism, and/or derivatives thereof and/or compounds related thereto, said process comprising:
obtaining an organism capable of producing compounds involved in lysine metabolism, derivatives thereof and/or compounds related thereto;
altering the organism; and
producing more compounds involved in lysine metabolism, and/or derivatives thereof, and/or compounds related thereto, by the altered organism as compared to the unaltered organism.

2. The process of claim 1 wherein the organism is C. necator or an organism with properties similar thereto.

3. The process of claim 1 wherein the organism is altered to express lysine decarboxylase with or without a PMD exporter system.

4. The process of claim 3 wherein the lysine decarboxylase is from E. coli.

5. The process of claim 3 wherein the lysine decarboxylase comprises SEQ ID NO:2 or a polypeptide with similar enzymatic activities exhibiting at least about 50% sequence identity to an amino acid sequence set forth in SEQ ID NO: 2 or a functional fragment thereof or is encoded by a nucleic acid sequence comprising SEQ ID NO:1 or a nucleic acid sequence encoding a polypeptide with similar enzymatic activities exhibiting at least about 50%, sequence identity to the nucleic acid sequence set forth in SEQ ID NO: 1 or a functional fragment thereof.

6. (canceled)

7. The process of claim 3 wherein the organism is altered to express a PMD exporter system.

8. The process of claim 7 wherein the PMD exporter system comprises a PMD antiporter or a PMD exporter.

9. The process of claim 8 wherein the PMD antiporter is from E. coli.

10. The process of claim 8 wherein the PMD antiporter comprises SEQ ID NO:4 or a polypeptide with similar enzymatic activities exhibiting at least about 50% sequence identity to an amino acid sequence set forth in SEQ ID NO: 4 or a functional fragment thereof or is encoded by a nucleic acid sequence comprising SEQ ID NO:3 or a nucleic acid sequence encoding a polypeptide with similar enzymatic activities exhibiting at least about 50% sequence identity to the nucleic acid sequence set forth in SEQ ID NO: 3 or a functional fragment thereof.

11. (canceled)

12. The process of claim 8 wherein the PMD exporter is from C. glutamicum.

13. The process of claim 8 wherein the PMD exporter comprises SEQ ID NO:6 or a polypeptide with similar enzymatic activities exhibiting at least about 50% sequence identity to an amino acid sequence set forth in SEQ ID NO: 6 or a functional fragment thereof or is encoded by a nucleic acid sequence comprising SEQ ID NO:5 or a nucleic acid sequence encoding a polypeptide with similar enzymatic activities exhibiting at least about 50% sequence identity to the nucleic acid sequence set forth in SEQ ID NO: 5 or a functional fragment thereof.

14. (canceled)

15. The process of claim 3 wherein the organism is further altered to express one or more of a putrescine oxidase, a putrescine transaminase and/or an aldehyde dehydrogenase.

16. The process of claim 15 wherein the putrescine oxidase is from R. jostii, the putrescine transaminase is from E. coli and/or the aldehyde dehydrogenase is from E. coli.

17. The process of claim 15 wherein the putrescine oxidase comprises SEQ ID NO:8 or a polypeptide with similar enzymatic activities exhibiting at least about 50% sequence identity to an amino acid sequence set forth in SEQ ID NO: 8 or a functional fragment thereof or is encoded by a nucleic acid sequence comprising SEQ ID NO:7 or a nucleic acid sequence encoding a polypeptide with similar enzymatic activities exhibiting at least about 50% sequence identity to the nucleic acid sequence set forth in SEQ ID NO: 7 or a functional fragment thereof.

18-19. (canceled)

20. The process of claim 15 wherein the putrescine transaminase comprises SEQ ID NO:10 or a polypeptide with similar enzymatic activities exhibiting at least about 50% sequence identity to an amino acid sequence set forth in SEQ ID NO: 10 or a functional fragment thereof or is encoded by a nucleic acid sequence comprising SEQ ID NO:9 or a nucleic acid sequence encoding a polypeptide with similar enzymatic activities exhibiting at least about 50% sequence identity to the nucleic acid sequence set forth in SEQ ID NO: 9 or a functional fragment thereof.

21-22. (canceled)

23. The process of claim 15 wherein the aldehyde dehydrogenase comprises SEQ ID NO:12 or a polypeptide with similar enzymatic activities exhibiting at least about 50% sequence identity to an amino acid sequence set forth in SEQ ID NO: 12 or a functional fragment thereof or is encoded by a nucleic acid sequence comprising SEQ ID NO:11 or a nucleic acid sequence encoding a polypeptide with similar enzymatic activities exhibiting at least about 50% sequence identity to the nucleic acid sequence set forth in SEQ ID NO:11 or a functional fragment thereof.

24. (canceled)

25. The process of claim 1 wherein the organism is further altered to eliminate phaCAB, involved in PHBs production and/or H16-A0006-9 encoding endonucleases thereby improving transformation efficiency.

26. (canceled)

27. An altered organism capable of producing more compounds involved in lysine metabolism, derivatives thereof and/or compounds related thereto as compared to an unaltered organism.

28. The altered organism of claim 27 which is C. necator or an organism with properties similar thereto.

29. The altered organism of claim 27 which expresses lysine decarboxylase with or without a PMD exporter system.

30. The altered organism of claim 29 wherein the lysine decarboxylase is from E. coli.

31. The altered organism of claim 29 wherein the lysine decarboxylase comprises SEQ ID NO:2 or a polypeptide with similar enzymatic activities exhibiting at least about 50% sequence identity to an amino acid sequence set forth in SEQ ID NO: 2 or a functional fragment thereof.

32. The altered organism of claim 29 wherein the lysine decarboxylase is encoded by a nucleic acid sequence comprising SEQ ID NO:1 or a nucleic acid sequence encoding a polypeptide with similar enzymatic activities exhibiting at least about 50% sequence identity to the nucleic acid sequence set forth in SEQ ID NO: 1 or a functional fragment thereof.

33. The altered organism of claim 29 which expresses a PMD exporter system.

34. The altered organism of claim 33 wherein the PMD exporter system comprises a PMD antiporter or a PMD exporter.

35. The altered organism of claim 34 wherein the PMD antiporter is from E. coli.

36. The altered organism of claim 34 wherein the PMD antiporter comprises SEQ ID NO:4 or a polypeptide with similar enzymatic activities exhibiting at least about 50% sequence identity to an amino acid sequence set forth in SEQ ID NO: 4 or a functional fragment thereof or is encoded by a nucleic acid sequence comprising SEQ ID NO:3 or a nucleic acid sequence encoding a polypeptide with similar enzymatic activities exhibiting at least about 50% sequence identity to the nucleic acid sequence set forth in SEQ ID NO: 3 or a functional fragment thereof.

37. (canceled)

38. The altered organism of claim 34 wherein the PMD exporter is from C. glutamicum.

39. The altered organism of claim 34 wherein the PMD exporter comprises SEQ ID NO:6 or a polypeptide with similar enzymatic activities exhibiting at least about 50% sequence identity to an amino acid sequence set forth in SEQ ID NO: 6 or a functional fragment thereof or is encoded by a nucleic acid sequence comprising SEQ ID NO:5 or a nucleic acid sequence encoding a polypeptide with similar enzymatic activities exhibiting at least about 50% sequence identity to the nucleic acid sequence set forth in SEQ ID NO: 5 or a functional fragment thereof.

40. (canceled)

41. The altered organism of claim 29 wherein the organism is further altered to express one or more of a putrescine oxidase, a putrescine transaminase and/or an aldehyde dehydrogenase.

42. The altered organism of claim 41 wherein the putrescine oxidase is from R. jostii, the putrescine transaminase is from E. coli and/or the aldehyde dehydrogenase is from E. coli.

43. The altered organism of claim 41 wherein the putrescine oxidase comprises SEQ ID NO:8 or a polypeptide with similar enzymatic activities exhibiting at least about 50% sequence identity to an amino acid sequence set forth in SEQ ID NO: 8 or a functional fragment thereof or is encoded by a nucleic acid sequence comprising SEQ ID NO:7 or a nucleic acid sequence encoding a polypeptide with similar enzymatic activities exhibiting at least about 50% sequence identity to the nucleic acid sequence set forth in SEQ ID NO: 7 or a functional fragment thereof.

44-45. (canceled)

46. The altered organism of claim 41 wherein the putrescine transaminase comprises SEQ ID NO:10 or a polypeptide with similar enzymatic activities exhibiting at least about 50% sequence identity to an amino acid sequence set forth in SEQ ID NO: 10 or a functional fragment thereof or is encoded by a nucleic acid sequence comprising SEQ ID NO:9 or a nucleic acid sequence encoding a polypeptide with similar enzymatic activities exhibiting at least about 50% sequence identity to the nucleic acid sequence set forth in SEQ ID NO: 9 or a functional fragment thereof.

47-48. (canceled)

49. The altered organism of claim 41 wherein the aldehyde dehydrogenase comprises SEQ ID NO:12 or a polypeptide with similar enzymatic activities exhibiting at least about 50% sequence identity to an amino acid sequence set forth in SEQ ID NO: 12 or a functional fragment thereof or is encoded by a nucleic acid sequence comprising SEQ ID NO:11 or a nucleic acid sequence encoding a polypeptide with similar enzymatic activities exhibiting at least about 50% sequence identity to the nucleic acid sequence set forth in SEQ ID NO:11 or a functional fragment thereof.

50. (canceled)

51. The altered organism of claim 27 wherein the organism is further altered to eliminate phaCAB, involved in PHBs production and/or H16-A0006-9 encoding endonucleases thereby improving transformation efficiency.

52. (canceled)

53. A bio-derived, bio-based, or fermentation-derived product produced from the method of claim 1, wherein said product comprises:
(i) a composition comprising at least one bio-derived, bio-based, or fermentation-derived compound or any combination thereof;
(ii) a bio-derived, bio-based, or fermentation-derived polyamides, nylons, polyurethanes, chelating agents, dietary supplements, proteins, topical medicaments and additives comprising the bio-derived, bio-based, or fermentation-derived composition or compound of (i), or any combination thereof;
(iii) a molded substance obtained by molding the bio-derived, bio-based, or fermentation-derived composition or compound of (i) or the bio-derived, bio-based, or fermentation-derived polyamides, nylons, polyurethanes, chelating agents, dietary supplements, proteins, topical medicaments and additives of (ii), or any combination thereof;
(iv) a bio-derived, bio-based, or fermentation-derived formulation comprising the bio-derived, bio-based, or fermentation-derived composition or compound of (i), the bio-derived, bio-based, or fermentation-derived polyamides, nylons, polyurethanes, chelating agents, dietary supplements, proteins, topical medicaments and additives of (ii), or the bio-derived, bio-based, or fermentation-derived molded substance of (iii), or any combination thereof; or
(v) a bio-derived, bio-based, or fermentation-derived semi-solid or a non-semi-solid stream, comprising the bio-derived, bio-based, or fermentation-derived composition or compound of (i), the bio-derived, bio-based, or fermentation-derived polyamides, nylons, polyurethanes, chelating agents, dietary supplements, proteins, topical medicaments and additives of (ii), the bio-derived, bio-based, or fermentation-derived formulation of (iii), or the bio-derived, bio-based, or fermentation-derived molded substance of (iv), or any combination thereof.

54. A bio-derived, bio-based or fermentation derived product produced in accordance with the central metabolism depicted in FIG. 1A, 1B 3 or 4.

55. An exogenous genetic molecule of the altered organism of claim 27.

56. The exogenous genetic molecule of claim 55 comprising a codon optimized nucleic acid sequence or an expression construct or synthetic operon for one or more of a lysine decarboxylase, a PMD exporter system, a putrescine oxidase, a putrescine transaminase and/or an aldehyde dehydrogenase.

57. The exogenous genetic molecule of claim 56 codon optimized for C. necator.

58. The exogenous genetic molecule of claim 55 comprising a codon optimized nucleic acid sequence encoding a lysine decarboxylase with or without a PMD exporter system.

59. The exogenous genetic molecule of claim 55 comprising a nucleic acid sequence encoding a lysine decarboxylase from E. coli.

60. The exogenous genetic molecule of claim 55 comprising SEQ ID NO:1 or a nucleic acid sequence encoding a polypeptide with similar enzymatic activities exhibiting at least about 50% sequence identity to the nucleic acid sequence set forth in SEQ ID NO: 1 or a functional fragment thereof.

61. The exogenous genetic molecule of claim 58 further comprising a nucleic acid sequence encoding a PMD exporter system.

62. The exogenous genetic molecule of claim 61 comprising a nucleic acid sequence encoding an E. coli PMD antiporter.

63. The exogenous genetic molecule of claim 61 wherein the nucleic acid sequence comprises SEQ ID NO:3 or a nucleic acid sequence encoding a polypeptide with similar enzymatic activities exhibiting at least about 50% sequence identity to the nucleic acid sequence set forth in SEQ ID NO: 3 or a functional fragment thereof.

64. The exogenous genetic molecule of claim 61 comprising a nucleic acid sequence encoding a C. glutamicum cg2893 PMD exporter.

65. The exogenous genetic molecule of claim 61 comprising SEQ ID NO:5 or a nucleic acid sequence encoding a polypeptide with similar enzymatic activities exhibiting at least about 50% sequence identity to the nucleic acid sequence set forth in SEQ ID NO: 5 or a functional fragment thereof.

66. The exogenous genetic molecule of claim 58 further comprising a nucleic acid sequence encoding one or more of a putrescine oxidase, a putrescine transaminase and/or an aldehyde dehydrogenase.

67. (canceled)

68. A process for the biosynthesis of compounds involved in lysine metabolism, and/or derivatives thereof and/or compounds related thereto, said process comprising providing a means capable of producing compounds involved in lysine metabolism, and/or derivatives thereof and/or compounds related thereto and producing compounds involved in lysine metabolism, and/or derivatives thereof and/or compounds related thereto with said means.

69. A process for biosynthesis of compounds involved in lysine metabolism, and derivatives thereof, and compounds related thereto, said process comprising:
a step for performing a function of altering an organism capable of producing compounds involved in lysine metabolism, derivatives thereof, and/or compounds related thereto such that the altered organism produces more compounds involved in lysine metabolism, derivatives thereof, and/or compounds compared to a corresponding unaltered organism; and
a step for performing a function of producing compounds involved in lysine metabolism, derivatives thereof, and/or compounds related thereto in the altered organism.

70-71. (canceled)


SEQUENCE LISTING (12 sequences)

SEQ ID NO: 1 - DNA, 2148 bp, Artificial sequence
  Synthetic
atgaacgtcatcgcgatcctgaaccacatgggcgtgtacttcaaggaagaacccatccgtgagctgcaccgcgccctggagcgcctgaactttcagatcgtgtacccgaacgaccgcgatgatctgctgaagctgatcgaaaacaatgcccgcctctgcggcgtcattttcgactgggacaagtacaacctggagctgtgcgaagagatcagcaagatgaacgaaaatctgcccctctacgcgttcgccaacacctattccacgctggacgtgagcctgaacgacctccgcctgcagatcagcttcttcgaatatgccctgggtgcggccgaggacatcgccaataagatcaagcagaccaccgacgagtatattaacacgatcctgccgccgctgacgaaggccctgttcaagtatgtgcgcgaaggtaagtacaccttttgcacgccgggccacatgggcggcaccgcgttccagaagtcgccggtgggctcgctgttctacgatttcttcggccccaacaccatgaagtcggacatctcgatctcggtgtcggagctgggctccctgctggaccactccggcccgcacaaggaagcggagcagtacatcgcgcgggtgttcaacgcggaccgcagctacatggtgaccaacggcacgtccaccgccaacaagatcgtgggcatgtacagcgccccggccggcagcacgattctcatcgaccggaactgccacaagtcgctgacccatctgatgatgatgagcgacgtgacgcccatctacttccgtcccacgcgcaatgcctacggcatcctgggcggcatcccccagtcggagttccagcacgccacgatcgccaagcgcgtgaaggaaaccccgaacgccacctggccggtgcatgccgtgatcacgaactcgacgtacgacggcctgctgtataacaccgactttatcaaaaagaccctcgacgtgaagtcgatccacttcgacagcgcgtgggtgccgtacacgaacttctccccgatttacgaaggcaagtgcggcatgtcgggtggccgcgtcgaaggcaaggtcatctacgaaacgcagagcacccacaagctcctggcggccttctcccaagccagcatgatccacgtcaagggcgatgtgaacgaagaaacgtttaacgaagcctacatgatgcataccaccacctcgccgcattatggtattgtggcgtccaccgaaaccgccgccgccatgatgaagggcaatgcgggcaagcgcctgatcaacggctccatcgagcgcgcgatcaagttccggaaggaaatcaagcgcctccgcaccgaaagcgacggctggttcttcgacgtgtggcagccggaccacatcgacaccacggagtgctggccgctgcgctcggattccacgtggcacggcttcaagaatatcgacaacgagcacatgtacctggacccgatcaaggtcaccctgctcaccccgggcatggaaaaggacggcaccatgagcgacttcggcatcccggccagcatcgtggccaagtacctggatgagcacggcatcgtggtggaaaagaccggcccctacaatctgctgttcctgttcagcatcggcatcgacaagaccaaggccctgtcgctgctccgtgcgctcacggatttcaagcgcgccttcgacctgaatctgcgcgtgaagaacatgctgccctcgctctatcgcgaggacccggagttctacgagaacatgcgcatccaagagctcgcgcagaacatccacaagctgatcgtgcatcacaatctgcccgatctcatgtatcgcgcgttcgaggtgctgccgacgatggtcatgaccccctatgcggcgttccaaaaggaactgcacggcatgaccgaagaagtctacctggacgagatggtcggccgcatcaacgccaacatgatcctgccgtacccgccgggcgtccccctggtcatgcccggcgaaatgatcacggaagaatcgcggccggtcctggagttcctgcagatgctgtgcgagatcggcgcccattaccccggcttcgaaaccgacatccacggcgcgtaccgccaggccgatggccggtataccgtgaaggtcctgaaggaagagtcgaagaagtga

SEQ ID NO: 2 - protein, 715 aa, E. coli
MNVIAILNHMGVYFKEEPIRELHRALERLNFQIVYPNDRDDLLKLIENNARLCGVIFDWDKYNLELCEEISKMNENLPLYAFANTYSTLDVSLNDLRLQISFFEYALGAAEDIANKIKQTTDEYINTILPPLTKALFKYVREGKYTFCTPGHMGGTAFQKSPVGSLFYDFFGPNTMKSDISISVSELGSLLDHSGPHKEAEQYIARVFNADRSYMVTNGTSTANKIVGMYSAPAGSTILIDRNCHKSLTHLMMMSDVTPIYFRPTRNAYGILGGIPQSEFQHATIAKRVKETPNATWPVHAVITNSTYDGLLYNTDFIKKTLDVKSIHFDSAWVPYTNFSPIYEGKCGMSGGRVEGKVIYETQSTHKLLAAFSQASMIHVKGDVNEETFNEAYMMHTTTSPHYGIVASTETAAAMMKGNAGKRLINGSIERAIKFRKEIKRLRTESDGWFFDVWQPDHIDTTECWPLRSDSTWHGFKNIDNEHMYLDPIKVTLLTPGMEKDGTMSDFGIPASIVAKYLDEHGIVVEKTGPYNLLFLFSIGIDKTKALSLLRALTDFKRAFDLNLRVKNMLPSLYREDPEFYENMRIQELAQNIHKLIVHHNLPDLMYRAFEVLPTMVMTPYAAFQKELHGMTEEVYLDEMVGRINANMILPYPPGVPLVMPGEMITEESRPVLEFLQMLCEIGAHYPGFETDIHGAYRQADGRYTVKVLKEESKK

SEQ ID NO: 3 - DNA, 1335 bp, Artificial sequence
  Synthetic
atgtcgtcggccaagaagattggcctcttcgcctgcacgggcgtcgtggccggcaatatgatgggcagcggtatcgccctgctcccggcgaatctggcctccatcggcggcatcgcgatctggggctggatcatctcgatcatcggcgccatgtcgctggcctacgtgtacgcccgcctggccacgaagaacccgcaacagggcggccccatcgcgtatgccggcgaaatcagcccggccttcggcttccagaccggcgtgctctactatcacgccaactggatcggcaacctggccatcggcattaccgcggtgtcgtacctgtccacgttcttccccgtcctgaacgacccggtgcccgccggtatcgcctgcatcgccatcgtgtgggtgttcacgttcgtcaacatgctgggtggcacgtgggtgagccgcctgaccaccattggcctcgtcctggtgctgatcccggtggtgatgaccgccatcgtcggctggcattggttcgacgccgcgacctatgcggcgaactggaacacggcggacaccacggacggccacgccatcattaagagcatcctgctgtgcctgtgggcgtttgtcggcgtggagtccgcggcggtcagcaccggcatggtgaagaatccgaagcgcacggtcccgctggccaccatgctgggcaccggcctcgccggcatcgtctacatcgcggcgacccaggtgctgtccggcatgtacccgtccagcgtcatggcggcgtcgggcgcccccttcgccatctcggcctcgacgatcctgggcaactgggccgcgccgctcgtgagcgcgtttaccgccttcgcctgcctgacctccctgggctcgtggatgatgctcgtgggccaggccggcgtccgcgcggccaacgacggcaacttccccaaggtctatggcgaagtcgattcgaacggcatcccgaagaagggtctgctgctggccgcggtcaagatgaccgcgctgatgatcctgatcaccctcatgaacagcgccggtggcaaggcgtcggatctgttcggcgagctgaccggcatcgcggtgctgctgaccatgctgccgtacttttactcgtgcgtggacctgatccgcttcgagggcgtgaacatccggaacttcgtgtccctgatctgcagcgtgctgggctgcgtgttctgcttcatcgccctgatgggcgcgtcgtcgttcgaactggccggcacgttcatcgtgagcctgatcatcctgatgttctacgcccgcaagatgcacgagcgtcagagccattcgatggacaaccacaccgcgagcaatgcccactga

SEQ ID NO: 4 - protein, 444 aa, E. coli
MSSAKKIGLFACTGVVAGNMMGSGIALLPANLASIGGIAIWGWIISIIGAMSLAYVYARLATKNPQQGGPIAYAGEISPAFGFQTGVLYYHANWIGNLAIGITAVSYLSTFFPVLNDPVPAGIACIAIVWVFTFVNMLGGTWVSRLTTIGLVLVLIPVVMTAIVGWHWFDAATYAANWNTADTTDGHAIIKSILLCLWAFVGVESAAVSTGMVKNPKRTVPLATMLGTGLAGIVYIAATQVLSGMYPSSVMAASGAPFAISASTILGNWAAPLVSAFTAFACLTSLGSWMMLVGQAGVRAANDGNFPKVYGEVDSNGIPKKGLLLAAVKMTALMILITLMNSAGGKASDLFGELTGIAVLLTMLPYFYSCVDLIRFEGVNIRNFVSLICSVLGCVFCFIALMGASSFELAGTFIVSLIILMFYARKMHERQSHSMDNHTASNAH

SEQ ID NO: 5 - DNA, 1485 bp, Artificial sequence
  Synthetic
atgacctcggaaacgctgcaggcccaggccccgaccaagacccagcgctgggccttcctggccgtgatctccggcggtctgttcctcatcggcgtggacaacagcatcctctacaccgccctgcccctgctgcgggagcagctcgcggcgaccgaaacccaggcgctgtggatcatcaatgcgtacccgctcctgatggccggcctgctgctgggcacgggcacgctgggcgacaagatcggccatcgccgcatgttcctgatgggcctgtcgatcttcggcatcgcgtcgctgggcgccgcctttgcgcccaccgcctgggcgctggtcgcggcccgggcgttcctgggcatcggtgcggccaccatgatgccggcgacgctggcgctgatccgtattaccttcgaagatgagcgcgagcgcaacaccgccatcggcatctggggctccgtggccatcctcggcgccgccgccggtccgatcatcggcggtgccctgctggagttcttctggtggggtagcgtgttcctgatcaatgtcccggtcgccgtgatcgccctgatcgccaccctgttcgtcgcccccgccaacatcgcgaacccgtccaagcactgggacttcctgtccagcttctacgcgctcctcacgctggcgggcctgatcatcacgatcaaggaaagcgtcaacaccgcccgccacatgccgctgctgctcggtgcggtgatcatgctgattatcggcgccgtcctgttcagctcgcgtcagaagaagatcgaggaacccctgctcgacctgtcgctgttccgcaaccgcctcttcctgggcggcgtggtggccgccggcatggccatgttcaccgtgtcgggcctggagatgacgacctcgcaacggtttcaactgtcggtgggcttcacgccgctggaagccggcctcctgatgatccccgcggccctgggcagcttcccgatgagcattatcggcggcgcgaacctccaccgctggggcttcaagccgctgatctcgggcggtttcgccgccaccgccgtgggcattgcgctgtgcatctggggcgccacgcacaccgatggcctgccgttctttatcgcgggcctgtttttcatgggcgcgggcgccggctccgtcatgtcggtgtcgagcaccgccatcatcggcagcgccccggtgcgcaaggccggcatggcgagctcgatcgaagaagtctcgtatgagtttggcaccctgctcagcgtggcgatcctgggcagcctgttccccttcttctactccctgcatgccccggccgaagtggccgacaatttctcggccggcgtccatcacgccatcgacggcgacgcggcccgcgcctcgctggacacggcgtatatcaacgtcctgatcattgcgctggtgtgcgccgtcgcggcggcgctcatctcctcctacctgttccgcggcaacccgaagggcgcgaacaacgcgcactga

SEQ ID NO: 6 - protein, 494 aa, C. glutamicum
MTSETLQAQAPTKTQRWAFLAVISGGLFLIGVDNSILYTALPLLREQLAATETQALWIINAYPLLMAGLLLGTGTLGDKIGHRRMFLMGLSIFGIASLGAAFAPTAWALVAARAFLGIGAATMMPATLALIRITFEDERERNTAIGIWGSVAILGAAAGPIIGGALLEFFWWGSVFLINVPVAVIALIATLFVAPANIANPSKHWDFLSSFYALLTLAGLIITIKESVNTARHMPLLLGAVIMLIIGAVLFSSRQKKIEEPLLDLSLFRNRLFLGGVVAAGMAMFTVSGLEMTTSQRFQLSVGFTPLEAGLLMIPAALGSFPMSIIGGANLHRWGFKPLISGGFAATAVGIALCIWGATHTDGLPFFIAGLFFMGAGAGSVMSVSSTAIIGSAPVRKAGMASSIEEVSYEFGTLLSVAILGSLFPFFYSLHAPAEVADNFSAGVHHAIDGDAARASLDTAYINVLIIALVCAVAAALISSYLFRGNPKGANNAH

SEQ ID NO: 7 - DNA, 1362 bp, Artificial sequence
  Synthetic
atgccgaccctgcagcgcgacgtggccatcgtcggcgccggcccgtcgggcctggccgccgccaccgcgctgcggaaggccggcctgtccgtggccgtcctggaagcccgggaccgcgtgggtggccgcacgtggaccgacaccatcgacggcgcgatgctggagatcggcggtcagtgggtgtcgcccgaccagaccgtgctcattagcctgctggacgagctgggcctggaaacgttcgatcgctatcgcgagggcgagtcggtgtacatctccgcctcgggcgagcgcacgcgctacaccggcgagtcctttcccgtcgatgaaacgacccgtaaggaaatggaccgcctgatccagatcctggacgacctggccgcccaggtcggcgccgaggaaccgtgggcccatccgctcgcgcgggagctggacacgatcagcttcaagcattggctgatcgagcagagcgacgacgccgaagcgcgcgacaacatcggcctgttcatcgcgggcggcatgctcaccaagcccgcgcattccttctcggcgctgcaggccgtcctgatggcggccagcgccggctcgttttcgcacctggtggatgaagatttcatcctggacaagcgcgtcatcggtggcatgcagcaagtgtcgatccgcatggccgcggcgctgggcgacgacgtgttcctgaatgcccccgtgcgtaccgtgcagtggtccgagaacggcgcggtggtcctggcggacggcgacatccgcgtcgaggcgagccgcgtcgtgctggccgtccccccgaacctctacagccgcatcagctatgatccgccgctgccgcgtcgccagcaccaaatgcaccagcaccagtcgctgggcctcgtgatcaaggtgcatgcggtctacgaaacgccgttctggcgcgaggacggcctcgccggcaccggcttcggcgcgtcggaagtcgtgcaagaagtctatgacaacacgaaccacgaggacacccggggtaccctggtggccttcgtgtcggacgaaaaggccgacgccatgttcgagctgtccgaagaagaacggcgtgccacgatcctgggctcgctcgcgcgctacctgggcccgaaggccgccgagccggtggtgtactacgagtcggactggggcagcgaggaatggacgcgcggcgcgtacgccgcgtcgttcgatctgggcggtctgcaccgctacggcaaggacacccgcaccccggtgggccccttccacttcagctgctccgatattgccgccgagggctatcagcacgtggacggcgcggtccgcatgggccagcgcaccgccgcggacatcgtggcccgcctgggcaagtga

SEQ ID NO: 8 - protein, 453 aa, R. jostii
MPTLQRDVAIVGAGPSGLAAATALRKAGLSVAVLEARDRVGGRTWTDTIDGAMLEIGGQWVSPDQTVLISLLDELGLETFDRYREGESVYISASGERTRYTGESFPVDETTRKEMDRLIQILDDLAAQVGAEEPWAHPLARELDTISFKHWLIEQSDDAEARDNIGLFIAGGMLTKPAHSFSALQAVLMAASAGSFSHLVDEDFILDKRVIGGMQQVSIRMAAALGDDVFLNAPVRTVQWSENGAVVLADGDIRVEASRVVLAVPPNLYSRISYDPPLPRRQHQMHQHQSLGLVIKVHAVYETPFWREDGLAGTGFGASEVVQEVYDNTNHEDTRGTLVAFVSDEKADAMFELSEEERRATILGSLARYLGPKAAEPVVYYESDWGSEEWTRGAYAASFDLGGLHRYGKDTRTPVGPFHFSCSDIAAEGYQHVDGAVRMGQRTAADIVARLGK

SEQ ID NO: 9 - DNA, 1380 bp, Artificial sequence
  Synthetic
atgaaccgcctgccgtcgtccgccagcgccctggcgtgctccgcgcacgccctcaacctgatcgagaagcgcacgctcgaccatgaagagatgaaggccctgaaccgcgaggtcattgagtatttcaaggaacacgtgaacccgggcttcctggagtaccgcaagagcgtgaccgccggtggcgactatggcgcggtcgagtggcaggccggctcgctgaacaccctggtggacacgcagggtcaggaatttatcgactgcctgggcggcttcggtatcttcaacgtcggccaccgcaacccggtggtcgtgtcggccgtccagaaccagctggccaagcagccgctccatagccaggaactgctggaccccctgcgcgccatgctggccaagaccctcgccgccctcacgccgggcaagctgaagtactcgttcttctgcaactcgggtaccgagtcggtggaagcggcgctgaagctggcgaaggcctaccagagcccgcgtggcaagtttaccttcatcgcgacgagcggcgccttccacggcaagtccctgggcgcgctgagcgcgaccgccaagtccacgttccgcaagcccttcatgccgctgctgccgggcttccggcatgtgccgttcggcaatatcgaggccatgcgcacggcgctgaatgagtgcaagaaaaccggcgacgacgtggccgcggtcattctggagccgatccagggcgagggcggcgtgatcctgccgccgcccggctacctgaccgcggtgcgtaagctgtgcgacgagttcggcgcgctgatgatcctggacgaagtgcagaccggcatgggccgcaccggcaagatgttcgcctgcgaacacgagaacgtgcagccggacatcctctgcctcgcgaaggccctgggcggtggcgtgatgcccatcggcgcgaccatcgcgaccgaagaagtgttttcggtcctgttcgacaaccccttcctgcacaccaccaccttcggcggcaacccgctggcgtgcgccgcggccctcgccacgatcaacgtcctgctggagcagaatctgccggcccaggccgaacaaaagggcgatatgctcctggatggcttccgccaactggcccgcgagtaccccgacctcgtccaagaagcgcgcggcaagggcatgctgatggcgatcgagttcgtggataacgaaatcggctacaacttcgcctcggagatgttccgtcagcgcgtgctggtcgccggcacgctgaacaatgccaagaccatccggatcgagcccccgctgacgctgaccatcgaacagtgcgagctggtcatcaaggccgcccgcaaggccctggcggcgatgcgcgtcagcgtggaagaagcctga

SEQ ID NO: 10 - protein, 459 aa, E. coli
MNRLPSSASALACSAHALNLIEKRTLDHEEMKALNREVIEYFKEHVNPGFLEYRKSVTAGGDYGAVEWQAGSLNTLVDTQGQEFIDCLGGFGIFNVGHRNPVVVSAVQNQLAKQPLHSQELLDPLRAMLAKTLAALTPGKLKYSFFCNSGTESVEAALKLAKAYQSPRGKFTFIATSGAFHGKSLGALSATAKSTFRKPFMPLLPGFRHVPFGNIEAMRTALNECKKTGDDVAAVILEPIQGEGGVILPPPGYLTAVRKLCDEFGALMILDEVQTGMGRTGKMFACEHENVQPDILCLAKALGGGVMPIGATIATEEVFSVLFDNPFLHTTTFGGNPLACAAALATINVLLEQNLPAQAEQKGDMLLDGFRQLAREYPDLVQEARGKGMLMAIEFVDNEIGYNFASEMFRQRVLVAGTLNNAKTIRIEPPLTLTIEQCELVIKAARKALAAMRVSVEEA

SEQ ID NO: 11 - DNA, 1488 bp, Artificial sequence
  Synthetic
atgaacttccaccacctggcgtactggcaggacaaggccctctccctcgccatcgagaaccggctgttcatcaacggcgagtacaccgccgccgccgagaatgaaaccttcgaaacggtggacccggtgacgcaggccccgctggccaagatcgcccgcggcaagtccgtcgatatcgaccgggccatgagcgccgcccggggtgtgttcgagcgcggcgactggagcctgagcagccccgcgaagcgcaaggccgtgctgaacaagctggccgacctcatggaagcgcatgcggaagaactggcgctgctggaaaccctggacacgggcaagccgattcgccacagcctgcgcgacgacattccgggcgccgcccgcgccatccgctggtatgcggaagcgatcgacaaggtgtatggcgaggtcgcgaccacgtcctcgcatgagctggccatgatcgtccgcgagcccgtcggcgtgatcgccgccatcgtgccctggaatttcccgctgctcctgacgtgctggaagctgggcccggcgctcgccgccggcaattcggtcatcctgaagccctcggagaagtcgccgctgtcggccatccgcctggcgggcctggccaaggaagccggcctgccggacggcgtcctgaacgtcgtcacgggcttcggtcatgaagccggccaggccctgagccgccacaatgacatcgacgcgattgccttcaccggctcgacccgcacgggtaagcaactgctgaaggacgcgggcgactcgaacatgaagcgcgtgtggctggaagcgggcggcaagagcgccaacatcgtgttcgccgactgcccggatctccagcaagcggccagcgccaccgcggcgggcatcttttacaaccagggccaagtctgcatcgcgggcacccgcctcctgctggaagagtcgatcgcggacgagttcctggccctgctgaagcagcaagcccagaactggcagccgggccacccgctggaccccgccaccacgatgggcaccctgatcgattgcgcgcacgcggattccgtccacagcttcatccgtgagggcgagtccaagggtcagctcctcctggacggccgcaacgccggcctggccgccgcgatcggcccgaccatcttcgtggatgtggacccgaacgcctcgctctcgcgcgaggaaatctttggcccggtgctggtcgtgacccgtttcaccagcgaggaacaggcgctgcagctggccaacgactcgcagtacggcctgggtgcggccgtgtggacccgtgatctgtcgcgcgcgcaccggatgtcgcgccgcctcaaggccggcagcgtgttcgtgaacaactacaacgacggcgacatgacggtgcccttcggcggctacaagcagagcggcaacggccgcgacaagtccctgcacgcgctggagaagttcaccgagctcaagaccatctggatctcgctggaagcgtga

SEQ ID NO: 12 - protein, 495 aa, E. coli
MNFHHLAYWQDKALSLAIENRLFINGEYTAAAENETFETVDPVTQAPLAKIARGKSVDIDRAMSAARGVFERGDWSLSSPAKRKAVLNKLADLMEAHAEELALLETLDTGKPIRHSLRDDIPGAARAIRWYAEAIDKVYGEVATTSSHELAMIVREPVGVIAAIVPWNFPLLLTCWKLGPALAAGNSVILKPSEKSPLSAIRLAGLAKEAGLPDGVLNVVTGFGHEAGQALSRHNDIDAIAFTGSTRTGKQLLKDAGDSNMKRVWLEAGGKSANIVFADCPDLQQAASATAAGIFYNQGQVCIAGTRLLLEESIADEFLALLKQQAQNWQPGHPLDPATTMGTLIDCAHADSVHSFIREGESKGQLLLDGRNAGLAAAIGPTIFVDVDPNASLSREEIFGPVLVVTRFTSEEQALQLANDSQYGLGAAVWTRDLSRAHRMSRRLKAGSVFVNNYNDGDMTVPFGGYKQSGNGRDKSLHALEKFTELKTIWISLEA